(19) United States
(12) Patent Application Publication
(10) Pub. No.: US 2005/0272787 A1
(43) Pub. Date: Dec. 8, 2005

(54) Process for preparing crystalline form A of valdecoxib

(76) Inventors: Eswaraiah Sajja, Hyderabad (IN); Anumula Raghupathi Reddy, Hyderabad (IN); Aalla Sampath, Karimnagar (IN); Gilla Goverdhan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/133,000

(22) Filed: May 19, 2005

Related U.S. Application Data
(60) Provisional application No. 60/572,597, filed on May 19, 2004.

(51) Int. Cl. C07D 261/06; A61K 31/42

(52) U.S. Cl. 514/378; 548/247

(57) ABSTRACT

Valdecoxib Form A is prepared by a process comprising adding aqueous ammonia to a solution of 4-(5-methyl-3-phenyl-4-isoxazolyl)benzenesulfonyl chloride in a halogenated hydrocarbon or water.

### CROSS-REFERENCE TO RELATED APPLICATION

This application derives priority from U.S. Provisional Application 60/572,597 filed May 19, 2004, the entire content of which is hereby incorporated by this reference.

### INTRODUCTION TO THE INVENTION

The present invention relates to a process for the preparation of crystalline Form A of valdecoxib. Valdecoxib, which is chemically known as 4-(5 methyl-3-phenyl-4-isoxazolyl) benzene sulfonamide, is represented by Formula (I)

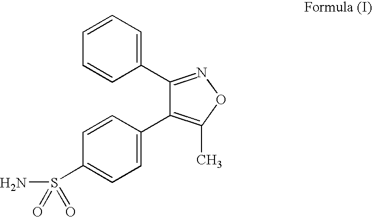

The present invention also relates to a process for the purification of 4-(5-methyl-3-phenyl-4-isoxazolyl) benzene sulfonyl chloride, an intermediate in the preparation of valdecoxib, and its subsequent conversion to valdecoxib. Valdecoxib obtained by this process is substantially free of its meta-isomer.

Valdecoxib is a potent and specific inhibitor of cyclooxygenase-2, and is used for the treatment of rheumatoid arthritis, osteoarthritis, and dysmenorrhea pain. A commercial pharmaceutical product containing valdecoxib has the trademark BEXTRA.

U.S. Pat. No. 5,633,272 discloses valdecoxib specifically, its pharmaceutical formulations and use in the treatment of rheumatoid arthritis, osteoarthritis, and dysmenorrhea pain. The '272 patent discloses a process for the preparation of valdecoxib, which comprises the reaction of desoxy benzoin with hydroxylamine hydrochloride in the presence of potassium hydroxide to give desoxybenzoin keto-oxime, which further reacts with acetic anhydride in the presence of N-butyl lithium in tetrahydrofuran to give 3,4-diphenyl-4-hydrido-5-hydroxy-5-methyl isoxazole. Successive treatment of the resultant solid with chlorosulfonic acid and saturated ammonium hydroxide solution gives 4-(5-methyl-3-phenyl isoxazol-4-yl) benzene sulfonamide.

U.S. Patent Application No. 2003/0162813 A1 also discloses a process for the preparation of valdecoxib, which comprises the reaction of 4-acetyl benzene sulfonyl chloride with ammonium hydroxide in ether to form 4-sulfonyl acetophenone, which on reaction with lithium diisopropylamide and hexamethyl phosphoramide yields 1-(4-sulfonyl phenyl)-1-propyne. Benzaldehyde oxime is reacted with chloramine-T in methanol and upon refluxing gives benzonitrile oxide. The resultant benzonitrile reacts with 1-(4-sulfonyl phenyl)-1-propyne in ethanol at reflux temperature to give 4-(5-methyl-3-phenyl-isoxazol-1-yl) benzene sulfonamide.

U.S. Pat. No. 6,441,014 discloses the process for the preparation of crystalline Form A (Example 6) and Form B (Example 1) of valdecoxib.

U.S. Patent Application No. 2003/0105334 A1 discloses a preparation of 4-(5-methyl-3-phenyl-4-isoxazolyl) benzene sulfonyl chloride in the presence of toluene and further recrystallization from heptane. The product has a purity of about 85 percent.

The processes disclosed in the art suffer from the disadvantages such as being costly, using pyrophoric reagents, and provide a poor yield, and are therefore not suitable for scale-up and commercial manufacturing.

Accordingly, there is a need for a process for the preparation of valdecoxib crystalline forms, which is simple, provides high yield, is industrially feasible, environmentally friendly and easy to scale-up.

### BRIEF DESCRIPTION OF THE DRAWING

FIG. 1 is an X-Ray powder diffraction (XRD) pattern of crystalline Form A of valdecoxib.

### SUMMARY OF THE INVENTION

The present invention relates to a process for the preparation of crystalline forms of valdecoxib, particularly Form A of valdecoxib.

One aspect of the present invention provides a process for the purification of 4-(5-methyl-3-phenyl-4-isoxazolyl) benzene sulfonyl chloride (an intermediate of valdecoxib) and its subsequent conversion to valdecoxib.

A process for the preparation of crystalline Form A of valdecoxib comprises:
(a) adding aqueous ammonia to a solution of 4-(5-methyl-3-phenyl-4-isoxazolyl) benzene sulfonyl chloride in a solvent;
(b) optionally adding seed crystals of Form A of valdecoxib to the reaction mass of step (a) and stirring;
(c) filtering the separated solid of step (b);
(d) washing the solid of step (c) by solvent of step (a); and
(e) drying the solid of step (d) to obtain crystalline Form A of valdecoxib.

A process for purifying 4-(5-methyl-3-phenyl-4-isoxazolyl) benzene sulfonyl chloride comprises:
(1) dissolving 4-(5-methyl-3-phenyl-4-isoxazolyl) benzene sulfonyl chloride in an organic solvent(s) accompanied by continuous stirring;
(2) cooling the solution of step (1) accompanied by stirring;
(3) separating the resultant solid by filtration;
(4) washing the solid with the solvent used in step (1); and
(5) drying the solid to obtain the pure 4-(5-methyl-3-phenyl-4-isoxazolyl) benzene sulfonyl chloride.

The processes of the present invention are simple, cost effective, industrially feasible, environmentally friendly, and commercially suitable over prior processes.

### DETAILED DESCRIPTION

One embodiment of the present invention is a process for the preparation of crystalline Form A of valdecoxib.

A process for the preparation of crystalline Form A of valdecoxib comprises:
(a) adding aqueous ammonia to a solution of 4-(5-methyl-3-phenyl-4-isoxazolyl) benzene sulfonyl chloride in a solvent;
(b) optionally adding seed crystals of Form A of valdecoxib to the reaction mass of step (a) accompanied by stirring to form a solid;
(c) separating the solid of step (b), such as by filtration;
(d) washing the solid of step (c) with the organic solvent of step (a);
(e) drying the solid of step (e) to obtain crystalline Form A of valdecoxib.

The solvents useful in the invention can be halogenated hydrocarbons such as dichloromethane, chloroform and dichloroethane, or mixtures thereof, or water. It is preferred to use dichloromethane or water.

The reaction is performed at temperatures below about 50° C., preferably about 25 to 35° C.

The quantity of solvent used in the above process can be varied from 5-30 times, preferably 10 times, of the weight of the 4-(5-methyl-3-phenyl-4-isoxazolyl) benzene sulfonyl chloride.

Crystalline Form A of valdecoxib as seeding material can be used optionally from about 2-10 percent by weight of the 4-(5-methyl-3-phenyl-4-isoxazolyl) benzene sulfonyl chloride.

Another embodiment of the present invention is a process for the purification of 4-(5-methyl-3-phenyl-4-isoxazolyl) benzene sulfonyl chloride (an intermediate of valdecoxib) and subsequent conversion of this intermediate to valdecoxib.

The process for the purification of 4-(5-methyl-3-phenyl-4-isoxazolyl) benzene sulfonyl chloride comprises:
(1) dissolving 4-(5-methyl-3-phenyl-4-isoxazolyl) benzene sulfonyl chloride in an organic solvent(s) accompanied by continuous stirring;
(2) cooling the solution of step (1), accompanied by stirring to form a solid;
(3) separating the resultant solid by filtration;
(4) washing the solid with solvent used in step (1);
(5) drying the solid to obtain the pure 4-(5-methyl-3-phenyl-4-isoxazolyl) benzene sulfonyl chloride.

The purified 4-(5-methyl-3-phenyl-4-isoxazolyl) benzene sulfonyl chloride can be dissolved in a solvent to form the starting solution for the process described above to prepare valdecoxib Form A.

Examples of useful organic solvents include cyclohexane, dichloromethane, dichloroethane, chloroform, ethyl acetate, acetone and hexane or mixtures thereof.

It is also possible to start the reaction using crude 4-(5-methyl-3-phenyl-4-isoxazolyl) benzene sulfonyl chloride. The obtained pure compound 4-(5-methyl-3-phenyl-4-isoxazolyl) benzene sulfonyl chloride obtained from the process can be converted to valdecoxib crystalline Form A which is substantially free from its meta isomer.

4-(5-methyl-3-phenyl-4-isoxazolyl) benzene sulfonyl chloride obtained by the present process generally has a purity of at least 99.5% by HPLC.

The processes of the present invention are further described by the following examples. These examples are provided for illustration only and should not be construed as a limitation of the scope of the invention.

### EXAMPLE 1

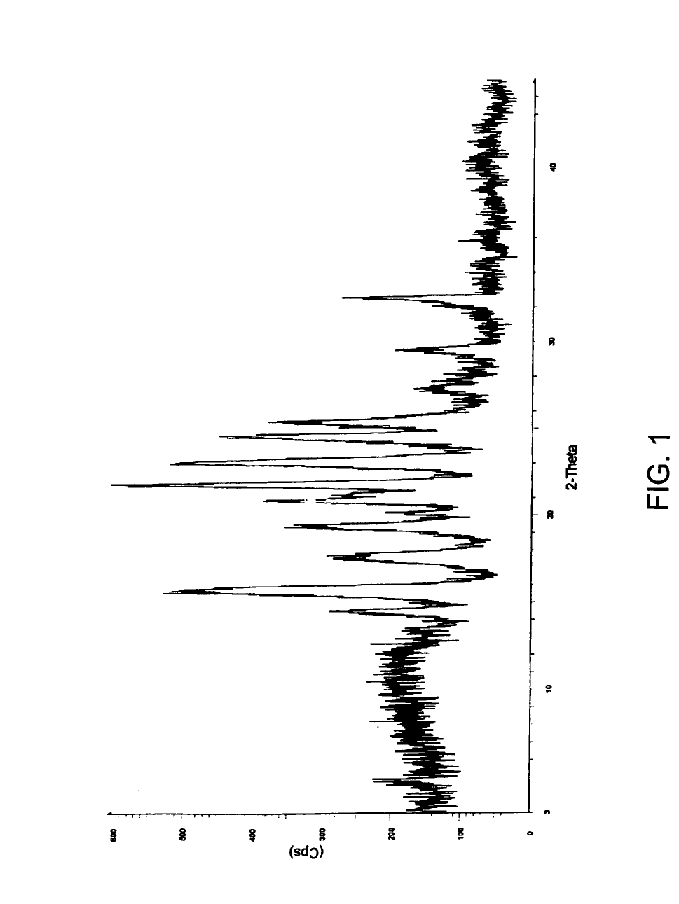

25 ml of 19.5% w/v aqueous ammonia solution were added slowly to a solution of 4-(5-methyl-3-phenyl-4-isoxazolyl) benzene sulfonyl chloride (5 grams) in dichloromethane (50 ml) at 25-30° C. 0.5 grams of crystalline Form A of valdecoxib (as seeding material) was charged to the reaction mass at 25-30° C. and the reaction mass was stirred for 35-45 minutes. The separated solid was filtered and washed with dichloromethane (5 ml). The compound was suction dried under reduced pressure followed by drying at 35-40° C. under reduced pressure to obtain the desired crystalline Form A of valdecoxib. The yield of the compound was 4 grams and the X-ray diffraction pattern for the product is shown in FIG. 1.

### EXAMPLE 2

25 ml of 19.5% w/v aqueous ammonia solution were added slowly to a solution of 4-(5-methyl-3-phenyl-4-isoxazolyl) benzene sulfonyl chloride (5 grams) in water (25 ml) at 25-35° C. The reaction mass was heated to 55-60° C. and stirred at this temperature until completion of the reaction. The reaction mass was cooled to about 20-30° C. The solid was filtered and washed with water (30 ml). The compound was suction dried under reduced pressure followed by drying at 25-35° C. under reduced pressure to obtain the desired crystalline Form A of valdecoxib. The yield of the compound was 4.2 grams.

### EXAMPLE 3

### PROCESS FOR PURIFICATION OF 4-(5-METHYL-3-PHENYL-4-ISOXAZOLYL) BENZENE SULFONYL CHLORIDE

5.0 grams of 4-(5-methyl-3-phenyl-4-isoxazolyl) benzene sulfonyl chloride were added to 5 ml of ethyl acetate and heated to 55-60° C. Charged 45 ml of cyclohexane and stirred at 55-60° C. for about 45 minutes. Cooled the reaction mass to 25-30° C. accompanied by stirring for 30-45 minutes. Filtered the solid and washed with 5 ml of cyclohexane. Dried the compound at 50° C. to get about 3.4 grams of the pure 4-(5-methyl-3-phenyl-4-isoxazolyl) benzene sulfonyl chloride.

### EXAMPLE 4

### PROCESS FOR PURIFICATION OF 4-(5-METHYL-3-PHENYL-4-ISOXAZOLYL) BENZENE SULFONYL CHLORIDE

5.0 grams of 4-(5-methyl-3-phenyl-4-isoxazolyl) benzene sulfonyl chloride were added to 5 ml of dichloromethane and heated to 35-45° C. and stirred for 30 minutes. 45 ml of cyclohexane was charged and the reaction mass was cooled to 25-30° C. accompanied by stirring for 30-45 minutes. The solid was filtered and washed with 5 ml of cyclohexane. The compound was dried at 50° C. to get the pure 4-(5-methyl-3-phenyl-4-isoxazolyl) benzene sulfonyl chloride. (Yield 3.9 grams).

### EXAMPLE 5

### PROCESS FOR PURIFICATION OF 4-(5-METHYL-3-PHENYL-4-ISOXAZOLYL) BENZENE SULFONYL CHLORIDE

5.0 grams of 4-(5-methyl-3-phenyl-4-isoxazolyl) benzene sulfonyl chloride were added to 50 ml of cyclohexane and heated to reflux temperature and stirred for 30 minutes. The reaction mass was cooled to 25-30° C. accompanied by stirring for 45-60 minutes. The solid was filtered and washed with 5 ml of cyclohexane. The resultant compound was dried at 50° C. to get the pure 4-(5-methyl-3-phenyl-4-isoxazolyl) benzene sulfonyl chloride. (Yield 3.9 grams).

### EXAMPLE 6

### PREPARATION OF CRYSTALLINE FORM-A OF VALDECOXIB FROM FORM B

Dissolve 100 gm of valdecoxib Form B in the mixture of 7.5 L of dichloromethane and 500 ml of aqueous ammonia under stirring. Separate the dichloromethane layer and distill off completely at 35° C. without stirring in a Rota Vapour flask. Dry the compound under reduced pressure at 35° C. in a Rota Vapour flask for 22-26 hours with rotation at 115-125 rpm. Further dry the compound at 100° C. under reduced pressure for 2.5 to 3.5 hours while rotating at 115-125 rpm. Cool to 25-35° C. and unload the compound, yielding 90.4 g of valdecoxib Form A (90.4%).

1. A process for preparing valdecoxib Form A crystals, comprising adding aqueous ammonia to a solution of 4-(5-methyl-3-phenyl-4-isoxazolyl)benzenesulfonyl chloride in a halogenated hydrocarbon or water.

2. The process of claim 1, wherein the solvent comprises a halogenated hydrocarbon.

3. The process of claim 1, wherein the solvent is a halogenated hydrocarbon comprising dichloromethane, chloroform, dichloroethane, or a mixture of any two or more thereof.

4. The process of claim 1, which is conducted at temperatures below about 50° C.

5. The process of claim 1, wherein 1 part of 4-(5-methyl-3-phenyl-4-isoxazolyl)benzenesulfonyl chloride is dissolved in about 5 to about 30 parts of solvent, by weight.

6. The process of claim 1, further comprising adding seed crystals of valdecoxib Form A to a mixture of aqueous ammonia and a solution of 4-(5-methyl-3-phenyl-4-isoxazolyl)benzenesulfonyl chloride.

7. The process of claim 6, wherein the weight of seed crystals comprises about 2 percent to about 10 percent of the weight of 4-(5-methyl-3-phenyl-4-isoxazolyl)benzenesulfonyl chloride.

8. A process for preparing valdecoxib Form A, comprising:
(a) dissolving 4-(5-methyl-3-phenyl-4-isoxazolyl) benzene sulfonyl chloride in an organic solvent;
(b) cooling the solution of step (a) to form a solid;
(c) isolating the solid;
(d) dissolving the solid in a halogenated hydrocarbon;
(e) adding aqueous ammonia to the solution; and
(f) isolating valdecoxib Form A.

9. The process of claim 8, wherein the organic solvent comprises cyclohexane, dichloromethane, dichloroethane, chloroform, ethyl acetate, acetone, hexane, or mixtures of any two or more thereof.

10. A process for purifying 4-(5-methyl-3-phenyl-4-isoxazolyl) benzene sulfonyl chloride, comprising:
(1) dissolving 4-(5-methyl-3-phenyl-4-isoxazolyl) benzene sulfonyl chloride in a solvent;
(2) cooling the solution; and
(3) isolating a solid.

11. The process of claim 10, wherein the solvent comprises cyclohexane, dichloromethane, dichloroethane, chloroform, ethyl acetate, acetone, hexane, or mixtures of any two or more thereof.

12. The process of claim 10, wherein the solvent comprises ethyl acetate, and the process further comprises adding cyclohexane to the solution before the cooling of step (2).

13. The process of claim 10, wherein the solvent comprises dichloromethane, and the process further comprises adding cyclohexane to the solution before the cooling of step (2).
